(19) United States
(12) Patent Application Publication
(10) Pub. No.: US 2005/0065552 A1
(43) Pub. Date: Mar. 24, 2005

(54) Medium or low frequency electro-stimulated massaging shoes

(76) Inventor: Tsung I. Yu, Taipei (TW)

Correspondence Address: BACON & THOMAS, PLLC, 625 SLATERS LANE, FOURTH FLOOR, ALEXANDRIA, VA, 22314, US

(21) Appl. No.: 10/667,659

(22) Filed: Sep. 23, 2003

(51) Int. Cl. A61N 1/04

(52) U.S. Cl. 607/2

(57) ABSTRACT

Medium or low frequency electro-stimulated massaging shoes comprises a pair of controllers and a pair of shoe bodies whose inner walls are provided with a plurality of conductive strips, each having a first end connected with at least one conductive plate and a second end connected with a conductive button. The conductive buttons of each of the shoe bodies are clustered on a predetermined location on the outer wall thereof, which can be coupled with a set of conductive buttons on a controller so as to mount the controller thereon. The controllers charge the conductive plates to provide the feet supported by the shoes with an electro-stimulated massage. Furthermore, an insulating shoe pad having a plurality of holes corresponding to foot acupuncture points is embedded within each shoe body, so that conductive pins may be inserted on predetermined holes thereof to massage a designated area on a foot.

### BACKGROUND OF THE INVENTION

(a) Field of the Invention

The present invention relates to medium or low frequency electro-stimulated massaging devices, and more particularly to a pair of medium or low frequency electro-stimulated massaging shoes, which is provided with a conductive button set for mounting an electro-stimulating controller thereon so as to achieve the effect of electro-stimulated foot massage.

(b) Description of the Prior Art

Footwear of the prior art is solely for providing a walk-assisting mechanism, which can be embodied in various forms such as slippers, sandals, sneakers, leather shoes and mountaineering boots. However, the conventional shoes cannot provide functions other than being a walk-assisting mechanism. There have been another type of shoes that provides a massaging effect, wherein a plurality of granules or magnets are deployed over the shoe pads for passively reacting a massage to feet supported thereon. Massaging shoes of this type is disadvantageous in that they cause discomforts to the users.

### SUMMARY OF THE INVENTION

Accordingly, the primary objective of the present invention is to provide a novel type of medium or low frequency electro-stimulated massaging shoes having a plurality of conductive strips on the inner wall of each shoe body. Each of the conductive strips has a first end connected with at least one conductive plate and a second end connected with a conductive button. The conductive buttons of a shoe body are clustered on a predetermined location on the outer wall thereof, which can be coupled with a corresponding set of conductive buttons on an electro-stimulating controller so as to buckle up the controller with the shoe body. The conductive plates are held in direct contact with the foot acupuncture points when the shoes are put on. Without the electro-stimulating controller, the shoes are ordinary shoes. When mounted onto the shoes, the electro-stimulating controller charges the conductive plates to provide the feet with the effect of a medium or low frequency electro-stimulated massage.

The secondary objective of the present invention is to provide a type of medium or low frequency electro-stimulated massaging shoes, wherein one of the shoe bodies is further provided with a connection socket to which the conductive strips therein are converged. A wire is then used to connect the connection socket and the electro-stimulating controller associated with another shoe body so that one controller simultaneously operates both shoe bodies.

It is a further objective of the present invention that an insulating shoe pad having a plurality of holes corresponding to foot acupuncture points is embedded on the lower inner wall within each shoe body. Therefore, one or more than one conductive pin can be inserted on a set of predetermined holes on the shoe pads, so that a designated area on a foot, according to the arrangement of the conductive pins, is massaged.

The various objects and advantages of the present invention will be more readily understood from the following detailed description when read in conjunction with the appended drawings.

### BRIEF DESCRIPTION OF THE DRAWINGS

FIG. 1 is a perspective view of the shoe bodies of the present invention.

FIG. 2 is a perspective view of the shoe bodies of the present invention with controllers mounted thereon.

FIG. 3 is a perspective view of the controllers of the present invention.

FIG. 4 is a cross-sectional view of a controller being buckling up with a shoe body.

FIG. 5 shows the connectivity of the central integrated circuit of the controller of the present invention.

FIG. 6 is the circuit layout of the controller of the present invention.

FIG. 7 is a bottom view of a shoe body of the present invention.

FIG. 8 is a cross-sectional side view of a shoe body of the present invention.

FIG. 9 is a local enlarged cross-sectional view of the upper shoe surface of a shoe body according to FIG. 8.

FIG. 10 is a perspective view of the present invention wherein the shoe bodies are in series connection with a controller.

FIG. 11 is a perspective view of the present invention wherein the shoe bodies are in parallel connection with a controller.

FIG. 12 is an exploded view of the conductive strips, conductive buttons and the shoe upper surface.

FIG. 13 is a perspective view of another preferred embodiment of the present invention, wherein an insulating shoe pad being attached onto a shoe body.

FIG. 14 is a top view of the insulating shoe pad in FIG. 13.

FIG. 15 is a top view of the acupuncture areas at the bottom of a foot.

FIG. 16 is a cross-sectional side view of a conductive pin after being inserted into a hole on an insulating shoe pad.

FIG. 17 is a cross-sectional side view of a conductive pin before being inserted into a hole on an insulating shoe pad.

### DETAILED DESCRIPTION OF THE PREFERRED EMBODIMENTS

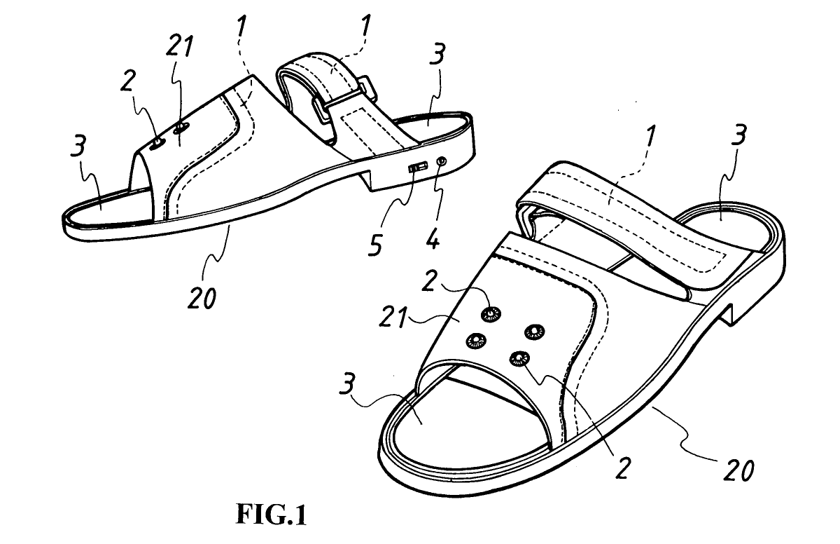

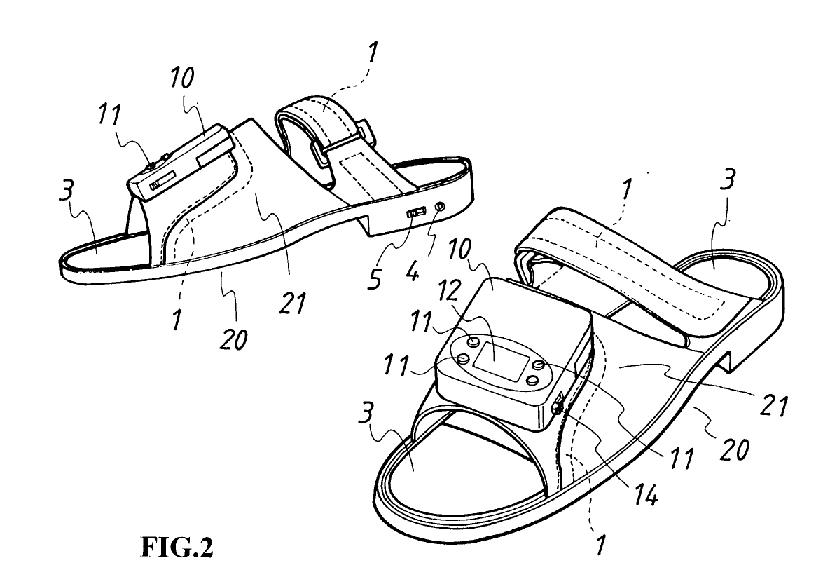

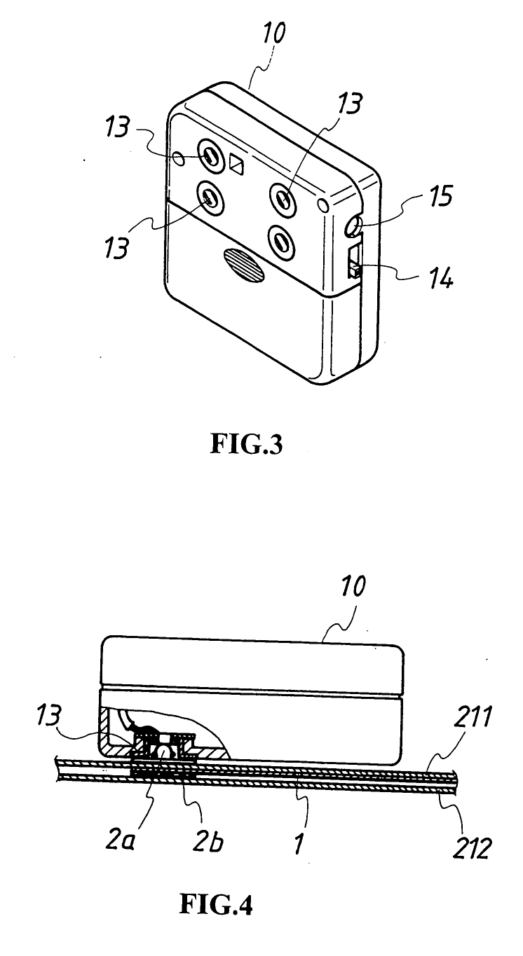

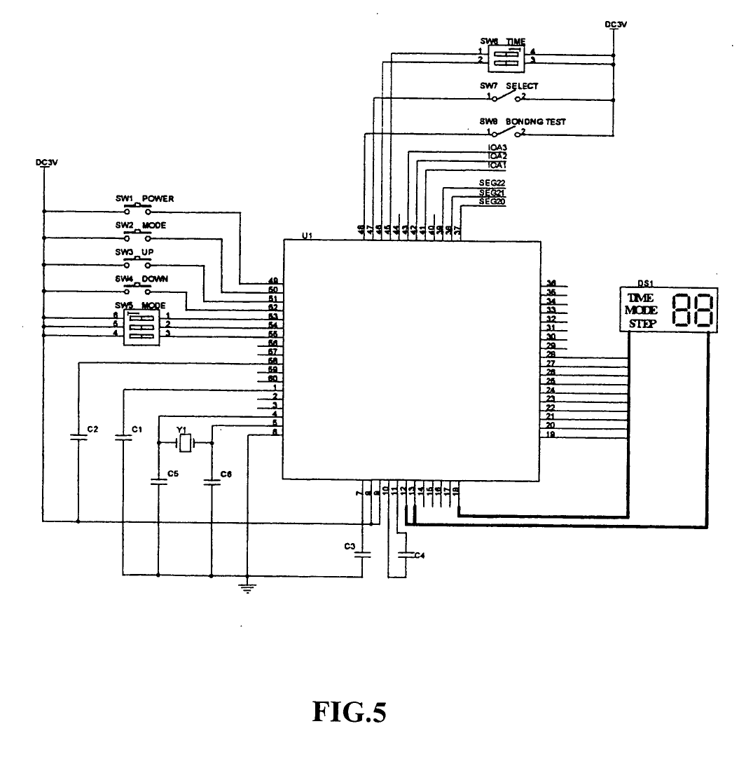

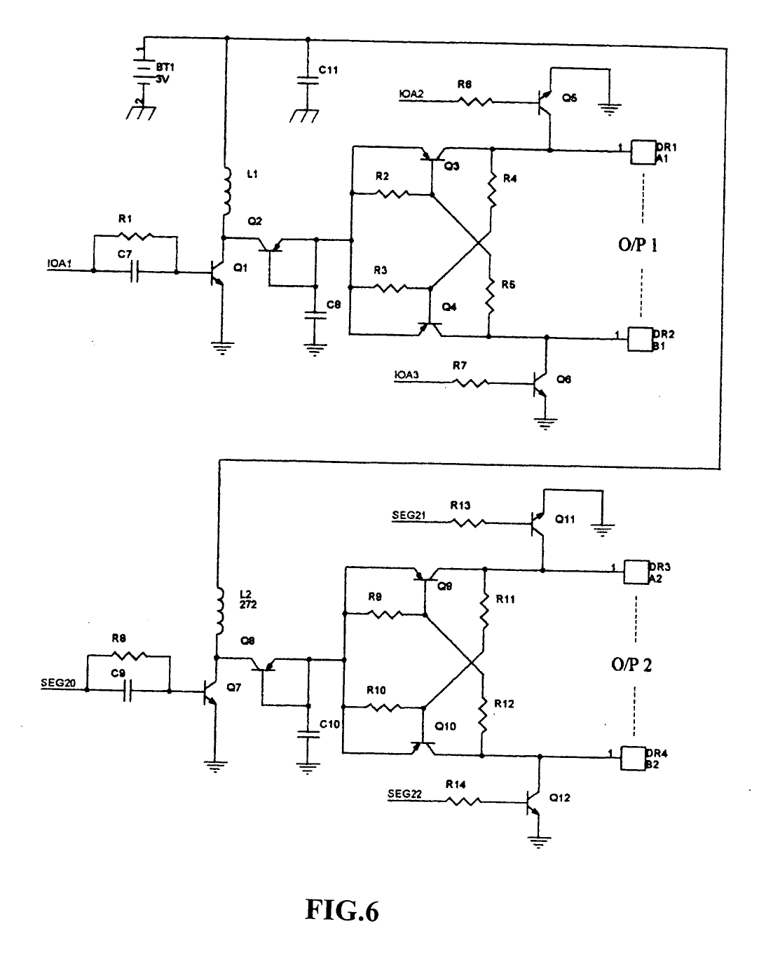

Referring to FIG. 1 and FIG. 2, medium or low frequency electro-stimulated massaging shoes according to the present invention comprises a controller 10 and a pair of shoe bodies 20. Referring to FIG. 3 to FIG. 6, the controller 10 is provided with a central integrated circuit (IC) and a charging/discharging circuit. The IC transports a pulsed-wave signal to the circuit for controlling the charging/discharging of the capacitors and inductors thereon from which a high voltage is generated for providing an electro-stimulating effect. The IC further adjusts the bandwidth of the pulsed-wave signal, in a range from 1 Hz to 150 Hz, to produce massaging effect of various strengths. Furthermore, the controller 10 has a plurality of control buttons 11 for respectively selecting current up/down, operation time, power on/off and massage mode. The controller 10 further contains an LCD display 12 for displaying the operation status. The pulsed high voltage generated by the internal circuit (as shown in FIG. 5 and FIG. 6) is connected to a plurality of conductive buttons 13, which then form an output terminal. A step switch 14 is installed on a lateral side of the controller 10 for selecting the charging region.

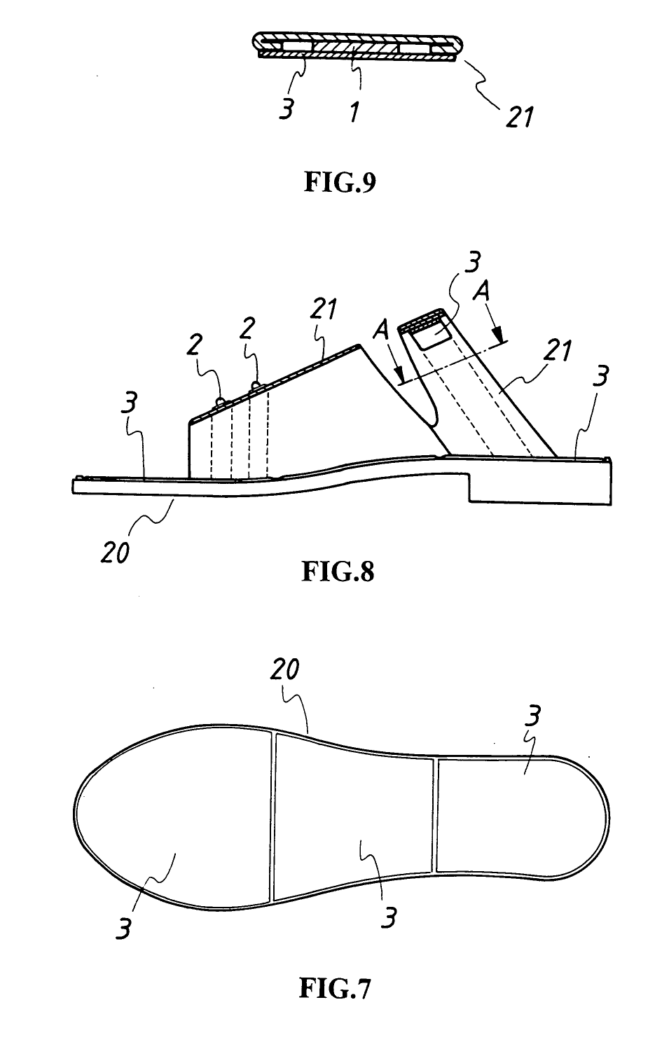

Referring to FIG. 7 and FIG. 9, the shoe bodies 20 are each composed of an upper portion and a lower portion, the inner walls of which are provided with a plurality of conductive strips 1. The conductive strips 1 are enclosed between the outer layer and the inner layer of the shoe surface 21 of respective shoe bodies, each having a first end connected with a conductive button 2 and a second end connected with a set of conductive plates 3. The conductive buttons 2 of each shoe body 20a or 20b are clustered on a pre-determined location of the shoe surfaces 21, which can be coupled with a corresponding set of conductive buttons 13 on the controller 10 so that each of the shoe surfaces 21 is mounted on with a controller 10, as shown in FIG. 4. Each conductive plate 3 is attached on a predetermined location on either the upper inner wall or the lower inner wall of a shoe body, which corresponds to an acupuncture point of a foot.

The above-mentioned shoe bodies 20 incorporate the ordinary functions of shoes and, as the shoe surfaces 21 thereof are each coupled with a controller 10, an additional function of providing a medium or low frequency electro-stimulated massage to the foot acupuncture points the charged conductive plates 3 are applied to.

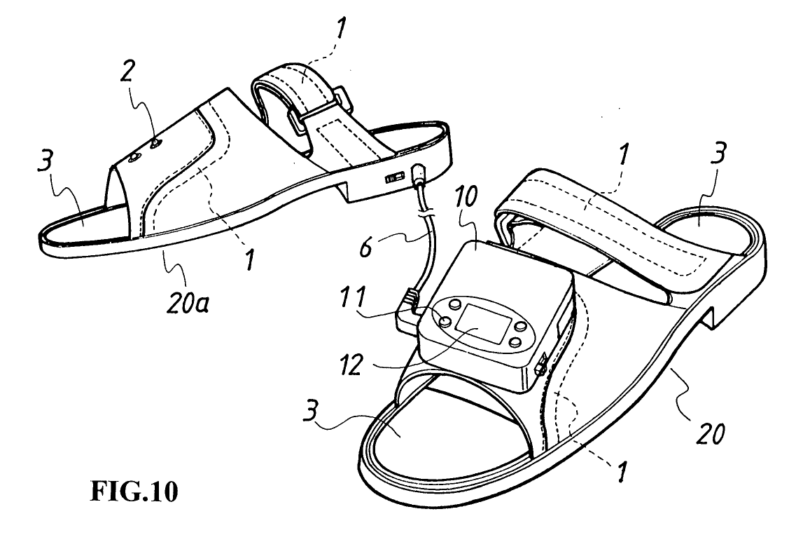

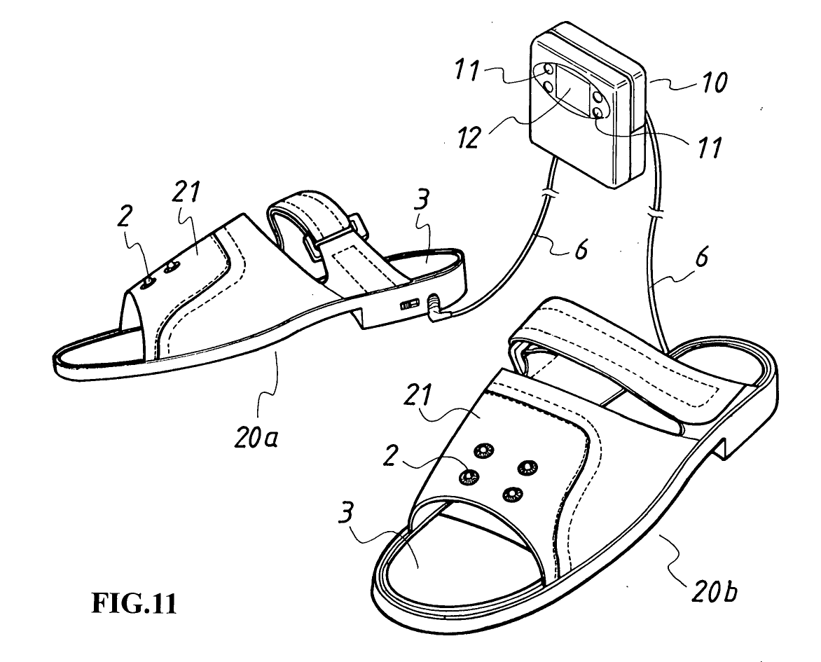

The above-mentioned controller 10 can be further provided with at least one connection port 15 for activating and controlling another set of conductive strips 1 and conductive plates 3 to produce an electro-stimulated massage effect thereon. Referring to the preferred embodiments in FIG. 10 or FIG. 11, the above-mentioned shoe bodies 20 may have a connection socket 4 and a step switch 5 formed on one of them, shoe body 20a in FIG. 10, or on both of them, shoe bodies 20a and 20b in FIG. 11. The connection sockets 4 are where the conductive strips 1 within the shoe bodies 20 converge to, and thereby a set of wires 6 can be used to connect the connection sockets 4, in series or in parallel, with the connection ports 15 on the controller 10. Therefore, a single controller 10 can simultaneously operate both of shoe bodies 20a and 20b to provide an electro-stimulated foot massage.

The above-mentioned step switches 14 and 5 on the controller 10 and on a shoe body 20a or 20b are for selecting a local foot area or the global foot area to get electro-stimulated, which is achieved by charging a selective portion of the conductive plates 3. Furthermore, the conductive strips 1 of the present invention are made of ordinary electro-conductive materials, and preferably soft electro-conductive cloths.

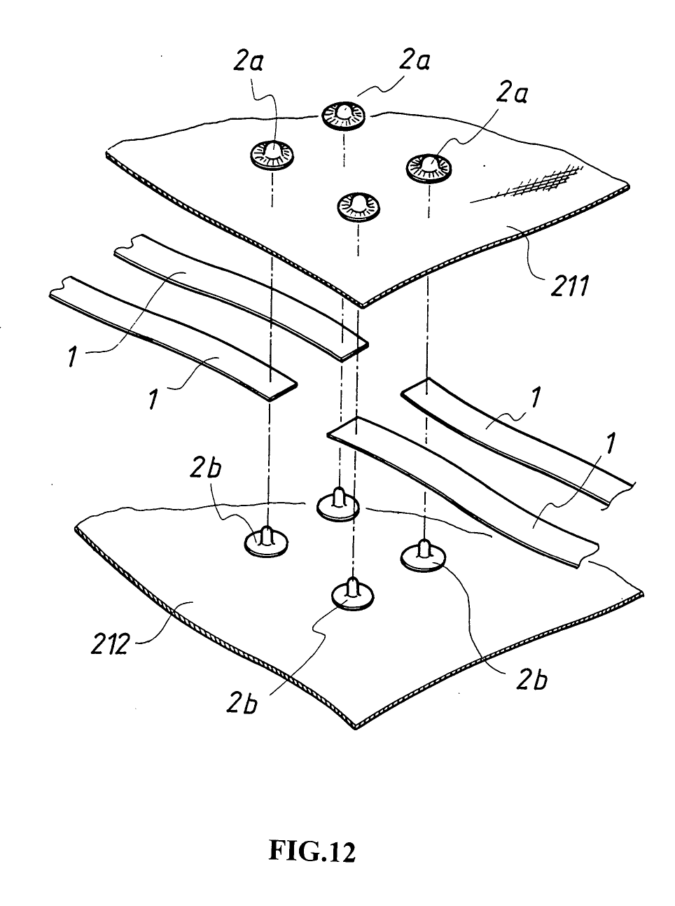

Referring to FIG. 12, the shoe surfaces 21 of the shoe bodies 20 are composed of an upper insulating cloth 211 and a lower insulating cloth 212, between which the conductive strips 1 are enclosed. The conductive buttons 2 at the first end of the conductive strips 1 are each composed of a female piece 2a and a male piece 2b, rivet-connected on two sides of the conductive strips 1. The conductive plates 3 are hidden on the lower or upper inner walls of the shoe bodies 20 between the upper insulating cloth 211 and the lower insulating cloth 212, except that the conductive buttons 2 are uncovered and bulged uprightly on the shoe surfaces 21.

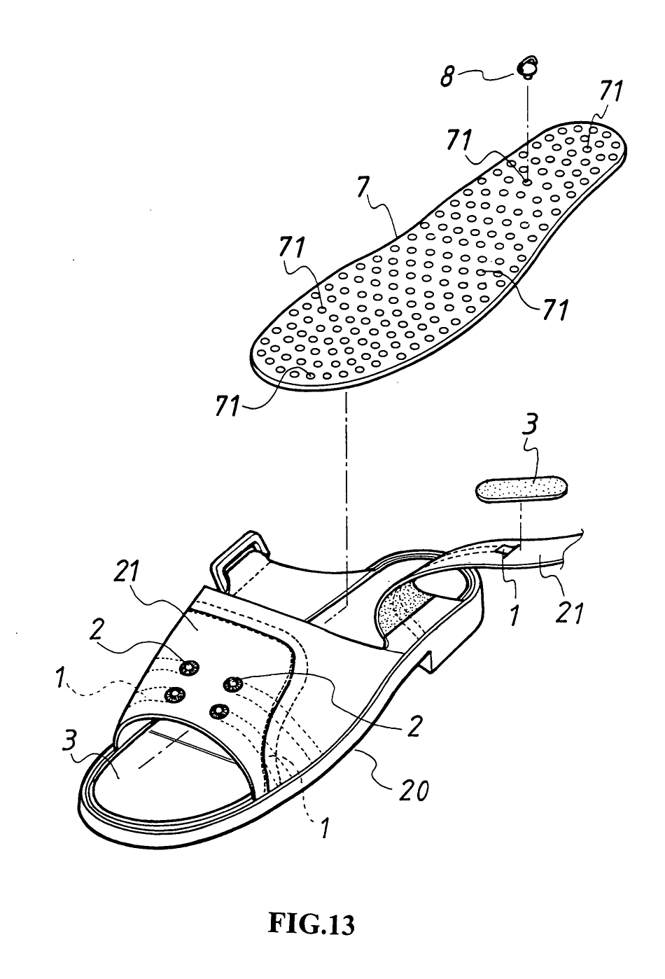

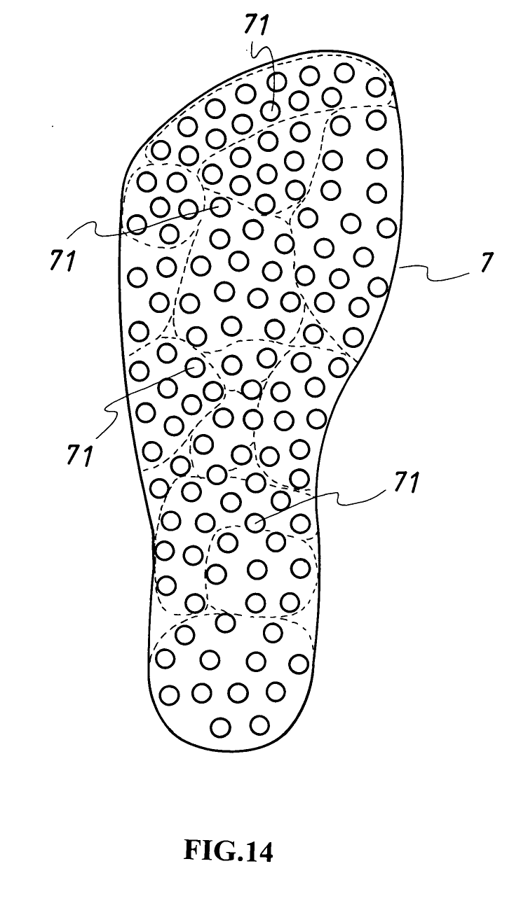

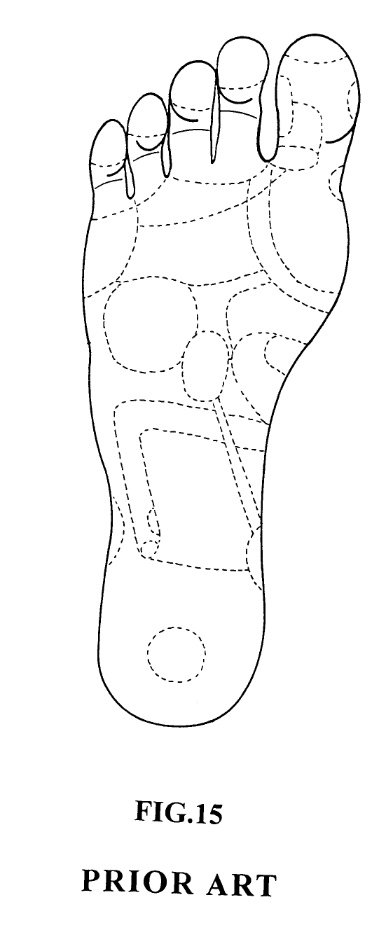

Referring to FIG. 13 and FIG. 14, the bottom inner surfaces of the shoe bodies 20, namely the lower inner walls, become an integral conductive area. An insulating shoe pad 7, which comprises a plurality of holes 71, is detachably or substantially attached onto the lower inner wall of each shoe body 20a or 20b. A number of adjacent holes 71 define an acupuncture area, which corresponds to an acupuncture portion at the bottom of a foot, as shown in FIG. 15. To activate an acupuncture area, the corresponding holes are inserted with a plurality of conductive pins 8. As a conductive shoe pad 7 is charged, the regions thereon with conductive pins 8 provide an electro-stimulated massage to the corresponding acupuncture portions of a foot, whereas those without conductive pins 8 are not conductive.

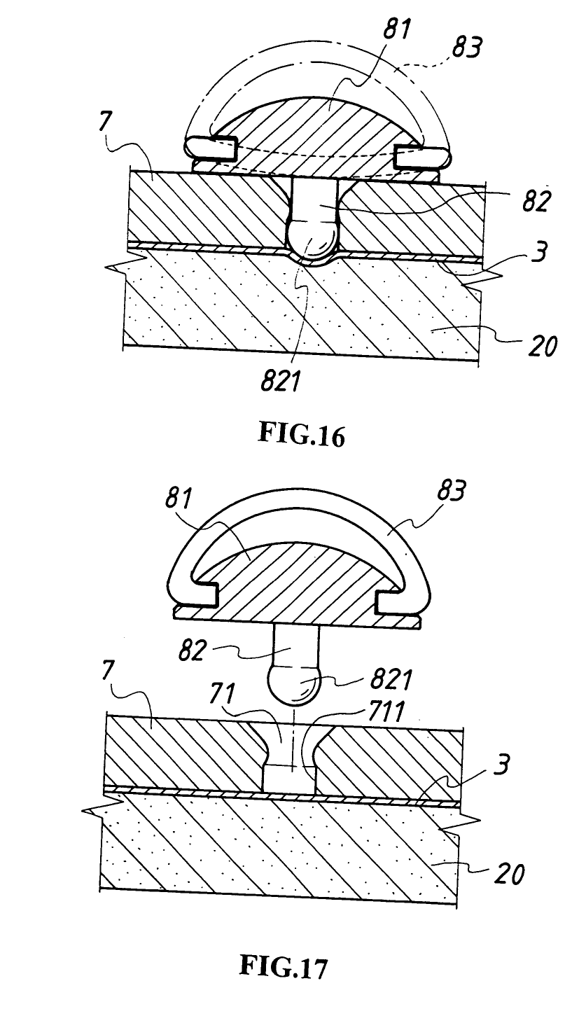

Referring to FIG. 16 and FIG. 17, the above-mentioned conductive pins 8 are made of conductive materials, each composed of an upper contact portion 81 and lower insertion portion 82. The insertion portion 82 is inserted through a hole 71 of an insulating shoe pad 7 to contact with the conductive plates 3, and therefore the contact portion 81, in contact with a foot, produces a massaging effect. It is a further mechanism that a pull ring 83 is pivotally connected with the contact portion 81 to facilitate the removal of a conductive pin 8 from a hole 71. The pull ring 83 can be folded aside to avoid contact with the foot that is supported on a shoe pad 7.

Furthermore, the insertion portion 82 of a conductive pin 8 has an enlarged head 821, and, correspondingly, a hole 71 has a shrunk opening 711 on the bottom face of the insulating shoe pad 7. Therefore, the insertion of a conductive pin 8 into an insulating shoe pad 7 is better secured.

1. A type of medium or low frequency electro-stimulated massaging shoes comprising:
a pair of controllers each for generating a pulsed high voltage, said pulsed high voltage being guided to a plurality of primary conductive buttons of each of said controllers, which then form an output terminal thereon; and
a pair of shoe bodies having a plurality of conductive strips deployed on an upper and a lower inner walls thereof, said conductive strips each having a first end connected with a secondary conductive button and a second end connected with a set of conductive plates, said secondary conductive buttons of each of said shoe bodies being clustered on a pre-determined location of a shoe upper surface of each of said shoe bodies, said secondary conductive buttons of each of said shoe bodies capable of being coupled with said primary conductive buttons on said controller so that each of said shoe bodies is mounted on with a controller, conductive plates of each of said shoe bodies being in contact with a foot housed therein;
said controllers charging the whole or a selective members of said conductive plates to exert a medium or low frequency electro-stimulated massage to foot portions said charged conductive plates applied to.

2. The medium or low frequency electro-stimulated massaging shoes of claim 1, wherein said controllers are each provided with a plurality of control buttons for respectively selecting current up/down, operation time and power on/off.

3. The medium or low frequency electro-stimulated massaging shoes of claim 1, wherein said controllers are each provided with an LCD panel for displaying operational status.

4. The medium or low frequency electro-stimulated massaging shoes of claim 1, wherein said controllers are each provided with a step switch for selecting members of said conductive plates to charge.

5. The medium or low frequency electro-stimulated massaging shoes of claim 1, wherein said conductive plates are forced to contact with acupuncture points of feet housed in said shoe bodies.

6. The medium or low frequency electro-stimulated massaging shoes of claim 1, wherein said controllers are provided with a connection port for connecting a peripheral device through a wire.

7. The medium or low frequency electro-stimulated massaging shoes of claim 1, wherein one of said shoe bodies is selected to install a connection socket to which said conductive strips converge.

8. The medium or low frequency electro-stimulated massaging shoes of claim 7, wherein said shoe body is provided with a step switch.

9. The medium or low frequency electro-stimulated massaging shoes of claim 1, wherein said conductive strips are made of electrically conductive cloths.

10. The medium or low frequency electro-stimulated massaging shoes of claim 1, wherein said shoe upper surfaces of said shoe bodies are each composed of an upper insulating cloth and a lower insulating cloth, between which said conductive strips are enclosed.

11. The medium or low frequency electro-stimulated massaging shoes of claim 1, wherein said conductive buttons at the first end of said conductive strips are each composed of a female piece and a male piece, rivet-connected on two sides of one said conductive strip; said conductive buttons are not covered by said insulating cloths on said shoe upper surfaces.

12. The medium or low frequency electro-stimulated massaging shoes of claim 1, wherein a bottom inner surface of each of shoe bodies becomes an integral conductive area and is covered with an insulating shoe pad; said insulating shoe pads each comprise a plurality of holes for at least one conductive pin to be inserted into.

13. The medium or low frequency electro-stimulated massaging shoes of claim 12, wherein said insulating shoe pads are detachably or substantially attached onto said bottom inner walls of said shoe bodies.

14. The medium or low frequency electro-stimulated massaging shoes of claim 12, wherein said conductive pins are made of conductive materials, each composed of an upper contact portion and lower insertion portion.

15. The medium or low frequency electro-stimulated massaging shoes of claim 14, wherein a pull ring is pivotally connected with said contact portion.

16. The medium or low frequency electro-stimulated massaging shoes of claim 14, wherein said conductive pins are each provided with an enlarged head, and, correspondingly, said holes on said insulating shoe pads are each provided with a shrunk opening on a bottom side thereof.
